(19) United States
(12) Patent Application Publication
(10) Pub. No.: US 2011/0004036 A1
(43) Pub. Date: Jan. 6, 2011

(54) HETEROGENEOUS DIMERIZATION OF ALPHA-OLEFINS WITH ACTIVATED METALLOCENE COMPLEXES

(75) Inventors: Tanja Englmann, Immenreuth (DE); Christine Denner, Gefrees (DE); Helmut G. Alt, Bayreuth (DE); Roland Schmidt, Bartlesville, OK (US)

Correspondence Address: CP / Baker & McKenzie LLP, Pennzoil Place, South Tower, 711 Louisiana, Suite 3400, Houston, TX, 77002, US

(73) Assignee: ConocoPhillips Company - IP Services Group, Houston, TX (US)

(21) Appl. No.: 12/829,057

(22) Filed: Jul. 1, 2010

Related U.S. Application Data
(60) Provisional application No. 61/222,452, filed on Jul. 1, 2009.

(51) Int. Cl. C07C 11/00; C07C 2/32

(52) U.S. Cl. 585/18; 585/512; 585/16

(57) ABSTRACT

Methods for dimerizing alpha-olefins utilizing immobilized buffered catalysts wherein the catalytic component is of the form (ligand)₂-M-X₂ where X is a halogen, M is selected from the group of Ti, Zr, and Hf and ligand is selected from the group consisting of cyclopentadienyl, substituted cyclopentadienyl, indenyl, and substituted indenyl, wherein the two ligands may be the same or different compound are provided.

### CROSS REFERENCE TO RELATED APPLICATIONS

This application claims priority to U.S. Provisional Application No. 61/222,452, filed Jul. 1, 2009.

### FEDERALLY SPONSORED RESEARCH STATEMENT

Not applicable.

### REFERENCE TO MICROFICHE APPENDIX

Not applicable.

### FIELD OF THE INVENTION

The invention relates to processes for dimerizing alkenes.

### BACKGROUND OF THE INVENTION

Dimerization of olefins is well known and industrially useful. Further, the use of transition metals to catalyze olefin dimerization and oligomerization is also known.

Use of ionic liquids for dimerization and oligomerization of olefins is also well known. In the broad sense, the term ionic liquids includes all molten salts, for instance, sodium chloride at temperatures higher than 800° C. Today, however, the term “ionic liquid” is commonly used for salts whose melting point is relatively low (below about 100° C.). One of the earlier known room temperature ionic liquids was [EtNH₃]+[NO₃] (m.p. 12° C.), the synthesis of which was published in 1914. Much later, series of ionic liquids based on mixtures of 1,3-dialkylimidazolium or 1-alkylpyridinium halides and trihalogenoaluminates, initially developed for use as electrolytes, were to follow.

One property of the imidazolium halogenoaluminate salts was that they were tuneable, i.e., viscosity, melting point and the acidity of the melt could be adjusted by changing the alkyl substituents and the ratio of imidazolium or pyridinium halide to halogenoaluminate. Imidazolium halogenoaluminate salts exhibit moisture sensitivity and, depending on the ratio of aluminum halide, Lewis acidic or Lewis basic properties. Ionic liquids with ‘neutral’, weakly coordinating anions such as hexafluorophosphate ([PF₆]⁻) and tetrafluoroborate ([BF₄]⁻) have also been used as alternatives to imidazolium halogenoaluminate salts. [PF₆]⁻ and [BF₄]⁻ based ionic liquids are generally highly toxic. Yet another anion for use in ionic liquids is bistriflimide [(CF₃SO₂)₂N]⁻, which does not exhibit the toxicity of [PF₆]⁻ and [BF₄]⁻ anions. Ionic liquids with less toxic cations are also known, including those with compounds like ammonium salts (such as choline) being used in lieu of imidazole.

Ionic liquids have found use as a catalyst in various chemical reactions. For example, Lewis acidic ionic liquids have been used as a catalyst to alkylate aromatic hydrocarbons, such as the alkylation of benzene with ethylene. In such processes, the ionic liquid itself serves as the catalyst, and the catalyst is neither buffered nor immobilized on a support. Ionic liquids have also been used in processes for making high viscosity polyalphaolefins using an oligomerization catalyst including an aluminum halide or alky-aluminum halides, and alkyl-substituted imidazolium halide or pyridinium halide. In such processes, the ionic liquid itself again serves as the catalyst and preferentially forms high-viscosity polyalphaolefins due to the lack of buffering.

Processes utilizing immobilized ionic liquids are also known. For example, immobilized ionic liquids may be prepared by functionalizing a support prior to contact with or forming the ionic liquid. Such known immobilized ionic liquids however are not buffered and therefore preferentially form high viscosity polyolefins. Again, in such systems, the ionic liquid itself functions as the catalyst.

Although all of the above methods are known and used in the synthesis of olefin oligomers and polymers, what is needed in the art is an improved synthetic method that allows for easy separation of the product. Especially in the case of olefin dimerizations, which usually yield liquids with relatively low viscosities or even gaseous di-olefins, the application of supported systems allowing the use of fixed bed reactors is superior to batch oligomerization, obviating the need for further product separation. In addition, the catalytically active surface may be maximized by use of high surface support materials, which optimizes the catalytic performance.

### SUMMARY OF THE INVENTION

Certain embodiments of the invention provide a process for the dimerization of olefins including: (1) modifying a support material containing —OH groups with an alkylaluminum compound to form a modified support material; (2) mixing an ionic liquid having a melting point below about 100° C. with an metallocene catalyst of the formula (ligand)₂-M-X₂ where X is a halogen, M is selected from the group of Ti, Zr, and Hf and ligand is selected from the group consisting of cyclopentadienyl, substituted cyclopentadienyl, indenyl, and substituted indenyl, wherein the two ligands may be the same or different compound, to form an ionic liquid/catalyst complex; (3) mixing the ionic liquid/catalyst complex with the modified support material to form an immobilized buffered catalyst; and (4) mixing the immobilized buffered catalyst with one or more alpha-olefins.

In specific embodiments of the invention the metallocene catalyst is selected from the following:

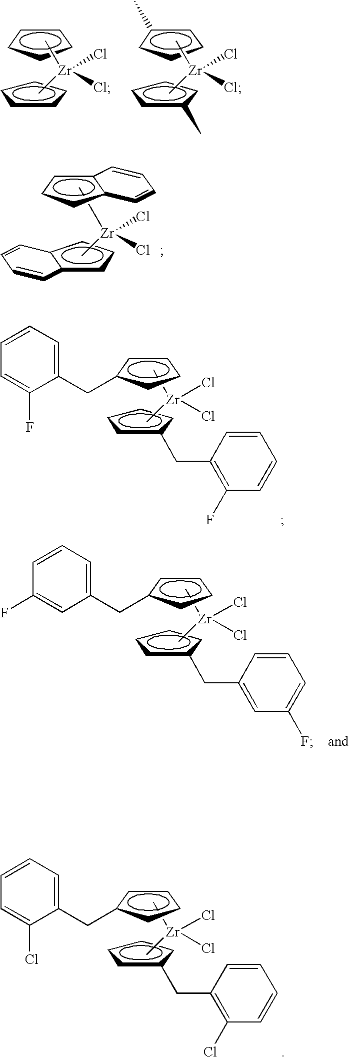

In yet other embodiments of the invention, the metallocene catalyst is bridged, for example, according to the formula:

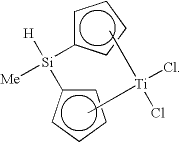

In some embodiments of the invention the one or more alpha-olefins are selected from the group alkenes having between three and ten carbon atoms.

In some embodiments, the ionic liquid comprises an anion selected from the group consisting of AlCl₃, AlRCl₂ and AlR₂Cl, where R is an alkyl chain. In yet specific embodiments of the invention, the ionic liquid comprises a cation selection from the group consisting of ammonium, imidazolium, sulfonium and phosphonium salts. In certain embodiments, the alkylaluminum compound is chloroethylaluminum.

Other embodiments of the invention provide a process for the dimerization of olefins including: (1) modifying a support material containing —OH groups with an alkylaluminum compound to form a modified support material; (2) mixing metallocene of the formula (ligand)₂-M-X₂ where X is a halogen, M is selected from the group of Ti, Zr, and Hf and ligand is selected from the group consisting of cyclopentadienyl, substituted cyclopentadienyl, indenyl, and substituted indenyl, wherein the two ligands may be the same or different compound, with one or more co-catalysts selected from the group of methylaluminoxane (“MAO”) and B(C₆F₅)₃ to form a combined catalyst; and (3) mixing the combined catalyst with one or more alpha-olefins.

### BRIEF DESCRIPTION OF THE DRAWINGS

FIG. 1 is a graphical representation showing the effect of time on the composition of product produced by an embodiment of the invention.

FIG. 2 illustrates nonlimiting examples of acceptable metallocene catalysts for use in the invention.

FIG. 3 is a graphical representation showing the effect of time on the composition of product produced by example 3.

### DESCRIPTION OF EMBODIMENTS OF THE INVENTION

In one embodiment of the invention, a process for dimerizing olefins utilizes a metallocene catalyst dissolved in a buffered ionic liquid immobilized on a support material.

For example, support material containing —OH groups may be modified with one or more aluminumhalide, alkylaluminumdihalide, and dialkylaluminumhalide or trialkylaluminum compounds (generically, “AlXₙR₃₋ₙ”). Generally, to achieve the support modification, the support material is mixed with a solution of the AlXₙR₃₋ₙ, with stirring. Suitable solvents include aromatics and paraffins, including halogenated paraffins, having 5 or more carbon atoms, including by way of example, toluene, benzene, pentane, hexane, cyclohexane and dichloromethane.

Excess solvent may be removed following a reaction time from between about 2 minutes to about 30 minutes, preferably between about 5 and 25 minutes and most preferably between about 10 and about 20 minutes. The result is a coated support material. Equation (1) below illustrates an exemplary formation of a modified support according to one embodiment of the invention.

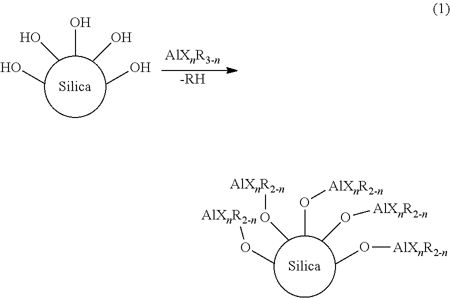

The ionic liquid is primarily a salt or mixture of salts that melt below room temperature. In some embodiments of the invention, the ionic liquid anion may be one or more of aluminum halide, alkylaluminum halide, gallium halide or alkylgallium halide. Preferably, the ionic liquid anion is one or more of AlCl₃, AlRCl₂, or AlR₂Cl where R is an alkyl chain. In some embodiments of the invention, the ionic liquid cation may be ammonium, imidazolium, sulfonium or phosphonium salt. In preferred embodiments, the ionic liquid cation is selected from ammonium halides containing one or more alkyl moieties having from 1 to about 9 carbon atoms, such as, for example, trimethylbenzylammoniumchloride, or hydrocarbyl substituted imidazolium halides, such as, for example, 1-butyl-3-methylimidazolium chloride.

The ionic liquid which will be used in producing the novel catalyst composition may be produced by first separately dissolving each of an acceptable cation and an acceptable anion in a solvent. The dissolved cation and anion are then mixed followed by removal of solvent.

In some embodiments of the invention, the ionic liquid is buffered. For example, a buffered system of ionic liquid may be produced utilizing one or more buffers having the general formula R₄Al₂Cl₂ or R₂Al₂Cl₄ where the neutral compounds are dissolved in an organic phase. For example:

2[Et₂Al₂Cl₅]⁻ [symbol] 2[AlCl₄]⁻+Et₄Al₂Cl₂

2[Et₂Al₂Cl₅]⁻ [symbol] 2[AlCl₄]⁻+Et₂Al₂Cl₄.

In some embodiments, the quarternary amine is dissolved first in methylene chloride and AlCl3 and stirred between 1 and 20 hours. The solvent is then removed by any of a number of known methods, most preferably by vacuum. The buffered system is then formed by addition of about 0.001 to about 0.2 equivalents Et₂AlCl to yield a ratio of the buffered system of amine:AlCl₃:Et₂AlCl of about 1:1.22:0.2.

A metallocene catalyst is then mixed with the ionic liquid. The metallocene catalyst has the general formula shown by equation (2) below:

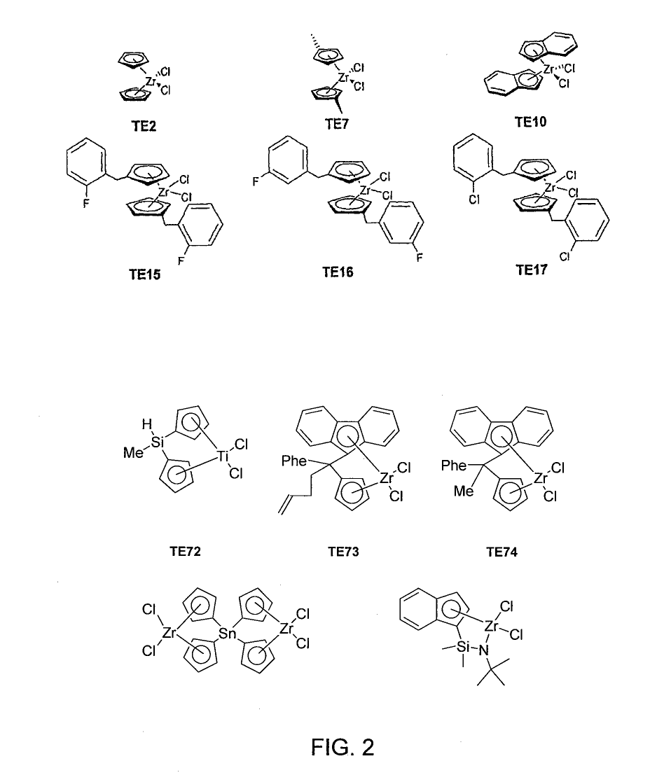

(ligand)₂-M-X₂ (2)

where M is a metal selected from the group consisting of Ti, Zr and Hf and X is a halogen. The ligand is selected from the group consisting of cyclopentadienyl, substituted cyclopentadienyl, indenyl, substituted indenyl, fluorenyl and substituted fluorenyl. Also useful in the present invention is a bridged metallocene catalyst. The two ligands could be bridged by way of an alkyl or alkenyl group having between 1 and 4 carbon atoms or silyl groups. In the alternative, the ligands can be bridged by a second metal, or alkylated metal wherein the metal is selected from the group consisting of Sn or Ge. The bridging unit may include one or two substituted groups, including alkyl, phenyl and other aryl groups. Nonlimiting examples of acceptable metallocene catalysts for use in the invention include the compounds shown in FIG. 2.

The immobilized buffered catalyst is then formed by mixing the metallocene catalyst/ionic liquid composition with the coated support material. Following sufficient mixing, excess solvent is removed, leaving an immobilized buffered catalyst solid or powder material. The immobilized buffered catalyst may then be mixed with one or more alpha-olefins to dimerize the olefins. In some embodiments, the immobilized buffered catalyst is mixed with a single alpha-olefin to form homogenous dimers or oligomers having fifty or fewer monomer units.

### Example 1

Ionic liquid preparation: 20.89 g 1-butyl-3-methylimidazolium chloride (“[BMIM]Cl”) 95%, BASF™) was dissolved in CH₂Cl₂. 19.43 g AlCl₃ (1.22 mol. eq., Reagent plus, SIGMA-ALDRICH™) was suspended in 100 ml CH₂Cl₂ and the suspension was slowly transferred to the solution of [BMIM]Cl. This addition was exothermic. The methylene chloride solvent was removed by subjecting the mixture to vacuum, heated to 70° C. and left on high vacuum until no more bubbles were observed. The result was a viscous, slightly colored liquid.

Catalyst solution preparation: Commercially available silica (DAVICAT® SI 1102 from W.R. GRACE & CO®) was calcined at 350° C. in dry argon for 4 hours. The calcined silica (3.0 g) was activated with methylaluminoxane (“MAO”) (10% in toluene). Metallocene catalyst TE2 (as illustrated in FIG. 2) is dissolved in toluene and activated with MAO in a ratio of 1:500. The activated TE2 catalyst was then added to the silica solution.

Dimerization of 1-hexene in a fixed bed reactor: The loaded silica (0.1-0.5 wt %, e.g. g Catalyst per g Silica, “loading ratio”) was then poured into the fixed bed reactor and flowed with 1:1 volume ratio of 1-hexene/toluene for 3 hours (flow ratio 1.0 ml/minute, WHSV (weight hourly space velocity) 748 1/h). Three samples were taken, having the composition, as determined by gas chromatography, as shown in Table 1 below.

TABLE 1

| | | | | tetra- | |
|---|---|---|---|---|---|
| Time | 1-hexene | dodecene | octadecene | eicosene | Oligomers > |
| (hours) | (wt %) | (wt %) | (wt %) | (wt %) | C₂₄ (wt %)* |
| 1 | 7.0 | 29.2 | 13.6 | 10.3 | 39.9 |
| 2 | 12.0 | 20.1 | 10.3 | 9.0 | 48.6 |
| 3 | 42.7 | 9.2 | 4.7 | 4.3 | 39.1 |

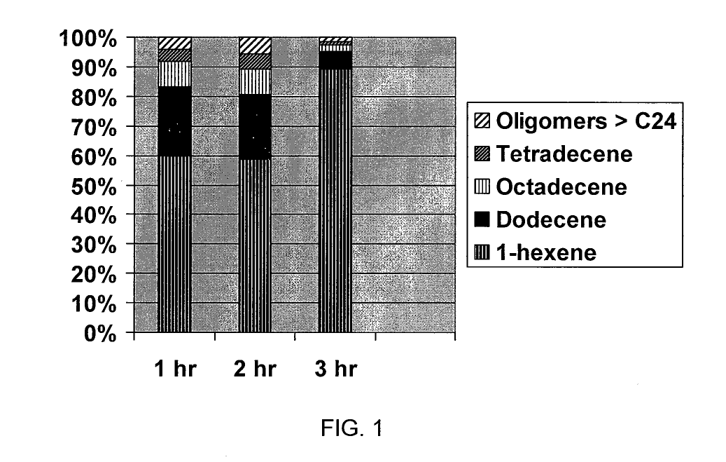

*Weight % of all oligomers having greater than twenty-four carbon atoms. FIG. 1 illustrates the effect of reaction time on product distribution. Specifically, the conversion of 1-hexene decreases within time.

### Comparative Example 1

Ionic liquid and activated catalyst TE2 were prepared as discussed in Example 1. Silica was alkylated with triethylaluminum (“AlEt₃”) (0.6 mmol AlEt₃/g silica). The catalyst solution was added to the silica. The fixed bed reaction with 1-hexene in propane was then conducted as described in Example 1. The resulting composition, as determined by gas chromatography, is shown in Table 2.

TABLE 2

| Time | 1-hexene | dodecene | octadecene |
|---|---|---|---|
| (hours) | (wt %) | (wt %) | (wt %) |
| 1 | 99.8 | 0.1 | 0.1 |
| 2 | 99.8 | 0.1 | 0.1 |
| 3 | 99.8 | 0.1 | 0.1 |

### Comparative Example 2

Ionic liquid and activated catalyst TE2 were prepared as discussed in Example 1. Commercially available silica (DAVICAT® SI 1102 from W.R. GRACE & CO®) was calcined at 350° C. in dry argon for 4 hours. The calcined silica is then added to the catalyst solution. The loaded silica (0.1-0.5% loading ratio) was then poured into the fixed bed reactor and flowed with 1:1 volume ratio of 1-hexene/toluene for 3 hours (flow ratio 0.5 ml/minute). The fixed bed reaction with 1-hexene in toluene was then conducted as described in Example 1. The foregoing procedure was also carried out using metallocene catalysts TE7, TE10, TE15, TE16 and TE17 (see FIG. 2). In all cases, with gas chromatography a conversion between 0 and 92% of the used 1-hexene was observed after 1 hour reaction time.

### Example 3

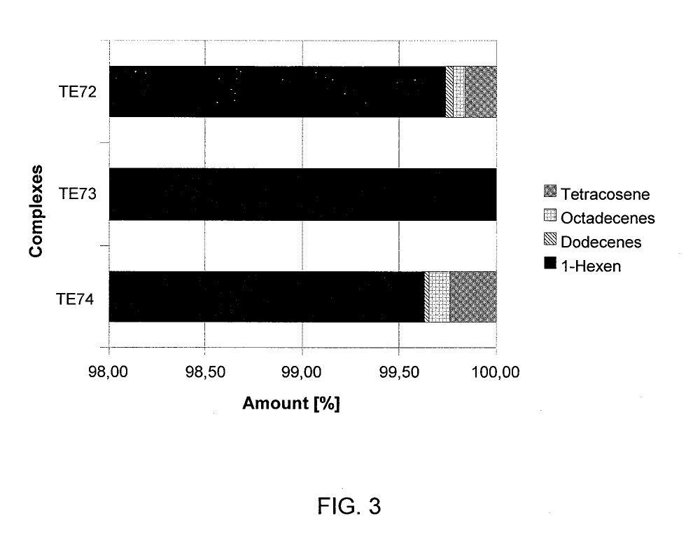

The bridged complexes TE72, TE73 and TE74 (shown in FIG. 2) are activated as discussed in Example 1. Commercially available silica (Kieselgel 60 from Macherey-Nagel) was calcined at 350° C. in dry argon for 4 hours. The calcined silica is then added to the catalyst solution and dried in vacuo. The loaded silica (0.1-0.5% loading ratio) was then poured into the fixed bed reactor and flowed with 1:1 volume ratio of 1-hexene/toluene for 3 hours (flow ratio 1.0 ml/minute). Every hour samples are taken and the products analyzed by GC. The calculated amounts after one hour are illustrated in FIG. 3.

The above examples are illustrative only and should not serve to unduly limit the invention.

What is claimed is:

1. A process for the dimerization of olefins comprising the steps of:
modifying a support material containing —OH groups with an alkylaluminum compound to form a modified support material;
mixing an ionic liquid having a melting point below about 100° C. with a buffer to form a buffered ionic liquid;
mixing the buffered ionic liquid with a metallocene catalyst of the formula (ligand)₂-M-X₂ where X is a halogen, M is selected from the group of Ti, Zr, and Hf and ligand is selected from the group consisting of cyclopentadienyl, substituted cyclopentadienyl, indenyl, and substituted indenyl, wherein the two ligands may be the same or different compound, to form a buffered ionic liquid/catalyst complex;
mixing the buffered ionic liquid/catalyst complex with the modified support material to form an immobilized buffered catalyst; and
mixing the immobilized buffered catalyst with one or more alpha-olefins.

2. The process of claim 1 wherein the metallocene catalyst has the formula:

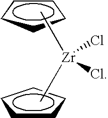

3. The process of claim 1 wherein the metallocene catalyst has the formula:

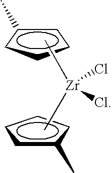

4. The process of claim 1 wherein the metallocene catalyst has the formula:

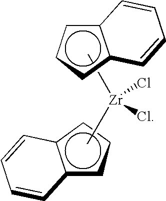

5. The process of claim 1 wherein the metallocene catalyst has the formula:

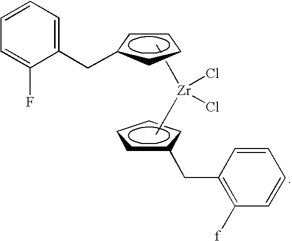

6. The process of claim 1 wherein the metallocene catalyst has the formula:

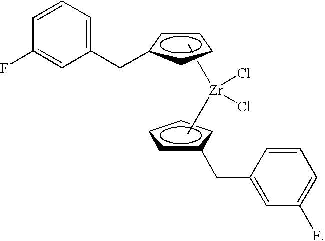

7. The process of claim 1 wherein the metallocene catalyst has the formula:

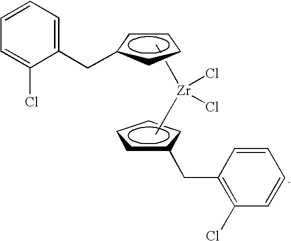

8. The process of claim 1 wherein the metallocene catalyst is bridged according to the formula:

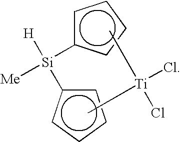

9. The process of claim 1 wherein the one of more alpha-olefins is selected from the group alkenes having between three and ten carbon atoms.

10. The process of claim 1 wherein the ionic liquid comprises an anion selected from the group consisting of AlCl₃, AlRCl₂ and AlR₂Cl, where R is an alkyl chain.

11. The process of claim 1 wherein the ionic liquid comprises a cation selection from the group consisting of ammonium, imidazolium, sulfonium and phosphonium salts.

12. The process of claim 1 wherein the alkylaluminum compound is chloroethylaluminum.

13. A process for the dimerization of olefins comprising the steps of:
modifying a support material containing —OH groups with an alkylaluminum compound to form a modified support material;
mixing a metallocene catalyst of the formula (ligand)₂-M-X₂ where X is a halogen, M is selected from the group of Ti, Zr, and Hf and ligand is selected from the group consisting of cyclopentadienyl, substituted cyclopentadienyl, indenyl, and substituted indenyl, wherein the two ligands may be the same or different compound, with one or more co-catalysts selected from the group of MAO and B(C₆F₅)₃ to form a combined catalyst; and
mixing the combined catalyst with one or more alpha-olefins.

14. A product comprising dimers of the one or more alpha-olefins produced using the process of claim 13.

15. The product of claim 14 wherein the one or more alpha-olefins are selected from C2-C20 alpha-olefins.

16. The process of claim 1 wherein the support material is silica.

17. The process of claim 13 wherein the support material is silica.

18. A product of the process of claim 1 wherein the product comprises dimers of the one or more alpha-olefins and oligomers having no more than fifty olefin units.

19. The product of claim 14 further comprising oligomers having no more than fifty olefin units.

20. The process of claim 13 wherein the metallocene catalyst is selected from the group of: [chemical structure]
